(19) United States
(12) Patent Application Publication
(10) Pub. No.: US 2008/0053446 A1
(43) Pub. Date: Mar. 6, 2008

(54) ADJUSTABLE CPAP MASK ASSEMBLY

(75) Inventors: Geoffrey P. Sleeper, Bay Village, OH (US); David Palkon, Tinley Park, IL (US)

Correspondence Address: PEARNE & GORDON LLP, 1801 EAST 9TH STREET, SUITE 1200, CLEVELAND, OH, 44114-3108, US

(73) Assignee: TIARA MEDICAL SYSTEMS, INC., Lakewood, OH (US)

(21) Appl. No.: 11/693,366

(22) Filed: Mar. 29, 2007

Related U.S. Application Data
(60) Provisional application No. 60/788,479, filed on Mar. 31, 2006.

(51) Int. Cl. A61M 16/06

(52) U.S. Cl. 128/205.25

(57) ABSTRACT

A respiratory mask assembly is provided, including a generally rigid mask frame coupled to a cushion formed of a resilient material. In one example, the cushion is maintained in abutment with the mask frame by a portion of a ledge being retained by a retainer member. In another example, the mask assembly further includes a collar for maintaining the cushion in abutment with the mask frame. Each of the mask frame, cushion, and collar can be generally triangularly shaped. The mask assembly can also include a plurality of generally triangularly-shaped cushions each including a relatively different face-engaging portion with an outer membrane. The mask assembly can also include a forehead support including a forehead support arm, forehead support flange, and a resilient forehead cushion. The forehead cushion is adapted to be infinitely vertically adjustable within a range relative to the forehead support arm.

### RELATED APPLICATIONS

This application claims the benefit of U.S. Provisional Application No. 60/788,479, filed on Mar. 31, 2006, the entire disclosure of which is hereby incorporated herein by reference.

### FIELD OF THE INVENTION

The present invention relates generally to ventilation devices, and more particularly, to an adjustable mask assembly for use in a continuous positive airway pressure system.

### BACKGROUND OF THE INVENTION

Sleep apnea is a potentially life-threatening breathing disorder characterized by brief interruptions of breathing during sleep. There are two types of sleep apnea: central and obstructive. Central sleep apnea, which is less common, occurs when the brain fails to send the appropriate signals to the breathing muscles to initiate respiration. Obstructive sleep apnea occurs when air cannot flow into or out of the person's nose or mouth although efforts to breathe continue. In a given night, the number of involuntary breathing pauses or “apneic events” may be as high as 20 to 60 or more per hour. Sleep apnea can also be characterized by choking sensations. The frequent interruptions of deep, restorative sleep often leads to excessive daytime sleepiness and may be associated with an early morning headache. Early recognition and treatment of sleep apnea is important because it may be associated with irregular heartbeat, high blood pressure, heart attack, and stroke.

Various forms of positive airway pressure during sleep can be an effective form of therapy for the apnea sufferer. Ventilation can be applied in the form of continuous positive airway pressure (CPAP), in which positive pressure is maintained in the airway throughout the respiratory cycle; bi-level positive airway pressure system, in which positive pressure is maintained during inspiration but reduced during expiration; and/or intermittent (non-continuous) positive pressure (IPPB), in which pressure is applied when an episode of apnea is sensed. In such procedures, a patient wears a mask over the mouth and/or nose during sleep, and pressure from an air blower forces air through the mouth and/or nasal passages. Typically, such masks receive a gas supply line that delivers gas into a chamber formed by wall of the mask and the patient's face. The walls are usually semi-rigid and have a face-contacting portion include an aperture that is aligned with the patient's mouth and/or nostrils. The face-contacting portion can include a soft, resilient elastomeric material that can conform to various facial contours. The mask is normally secured to the patient's head by straps. The straps are adjusted to pull the mask against the face with sufficient force to achieve a gas tight seal between the mask and the patient's face but not so tight as to be uncomfortable. Gas is thus delivered to the mask and into the patient's mouth and/or nasal passages.

Problems often arise with masks of the above configuration. For example, the face-contacting portion may apply excessive pressure to the wearer's face resulting in discomfort and possibly skin irritation. This can occur because the face-contacting portion has to distort beyond its normal range of elasticity to conform to certain facial contours, which requires the application of excessive forces. In some cases these excessive pressures and forces may cause the face to distort to conform to the face-contacting portion, which can increase wearer discomfort, resulting in facial soreness and ulceration.

### BRIEF SUMMARY OF THE INVENTION

The following presents a simplified summary of the invention in order to provide a basic understanding of some aspects of the invention. This summary is not an extensive overview of the invention. It is intended to identify neither key nor critical elements of the invention nor delineate the scope of the invention. Its sole purpose is to present some concepts of the invention in a simplified form as a prelude to the more detailed description that is presented later.

In accordance with an aspect of the present invention, a respiratory mask assembly is provided including a generally rigid mask frame having a generally triangularly-shaped outer periphery. The mask frame further includes a wall extending from the outer periphery of the frame and including an aperture, and a shelf extending inwardly from the wall and including a retainer member extending therefrom. The mask assembly further includes a generally triangularly-shaped cushion formed of a resilient material having a frame-engaging portion connected by a sidewall to a face-engaging portion adapted to sealingly connect the respiratory mask to a wearer's face. The frame-engaging portion includes a ledge extending a distance inwardly from the sidewall and a lug extending a distance outwardly from the sidewall. The frame-engaging portion is supported by the shelf and is maintained in abutment therewith by a portion of the ledge being retained by the retainer member and the lug being engaged with the aperture of the frame.

In accordance with another aspect of the present invention, a respiratory mask assembly is provided including a generally rigid mask frame having a generally triangularly-shaped outer periphery. The mask frame also includes a wall extending from the outer periphery of the frame and including an aperture, and a shelf extending inwardly from the wall and including a retainer member extending therefrom. The mask assembly further includes a generally triangularly-shaped cushion formed of a resilient material having a frame-engaging portion connected by a sidewall to a face-engaging portion adapted to sealingly connect the respiratory mask to a wearer's face. The frame-engaging portion is supported by the shelf and further includes a ledge. The mask assembly further includes a generally rigid, generally triangularly-shaped collar including a side member and a top member extending a distance outwardly therefrom. The side member includes an anchor, the top member is engaged with the ledge and the anchor is engaged with the retainer member to sealingly connect the cushion to the mask frame.

In accordance with another aspect of the present invention, a respiratory mask assembly is provided including a generally rigid mask frame having a generally triangularly-shaped outer periphery, a generally rigid, generally triangularly-shaped collar engageable with the mask frame, and a plurality of generally triangularly-shaped cushions being formed of a resilient material. Each cushion includes a face-engaging portion with an outer membrane. The collar is adapted to sealingly connect the cushions to the mask frame such that each of the cushions is alternatively engageable with the mask frame.

In accordance with another aspect of the present invention, a respiratory mask assembly is provided including a generally rigid mask frame, a cushion formed of a resilient material supported by the mask frame and adapted to sealingly connect the respiratory mask to a wearer's face, and a forehead support including a forehead support arm coupled to the mask frame. A forehead support flange is coupled to the forehead support arm, and a resilient forehead cushion is coupled to the support flange. The forehead cushion is adapted to be infinitely vertically adjustable within a range relative to the forehead support arm.

### BRIEF DESCRIPTION OF THE DRAWINGS

The foregoing and other features and advantages of the present invention will become apparent to those skilled in the art to which the present invention relates upon reading the following description with reference to the accompanying drawings, in which:

FIG. 1A illustrates a side view of an example adjustable CPAP mask assembly in accordance with an aspect of the present invention;

FIG. 1B is similar to FIG. 1A, but shows an alternate forehead cushion in accordance with another aspect of the present invention;

FIG. 2 illustrates a back view of the adjustable CPAP mask assembly of FIG. 1A in accordance with an aspect of the present invention;

FIG. 3 illustrates a front view of another example adjustable CPAP mask assembly in accordance with another aspect of the present invention;

FIG. 4 illustrates a side view of the adjustable CPAP mask assembly of FIG. 3;

FIG. 5 illustrates a perspective back view of the adjustable CPAP mask assembly of FIG. 3;

FIG. 6 illustrates a perspective front view of the adjustable CPAP mask assembly of FIG. 3 showing an example connection between a face cushion and a mask frame;

FIG. 7 illustrates a perspective, exploded view of the adjustable CPAP mask assembly of FIG. 6;

FIG. 8 illustrates a front view of an example collar in accordance with another aspect of the present invention;

FIG. 9 illustrates a sectional view of the adjustable CPAP mask assembly along line 9-9 of FIG. 1A;

FIG. 10 is similar to FIG. 9, but omits the face cushion;

FIG. 11A illustrates a front view of an example large size face cushion in accordance with another aspect of the present invention;

FIG. 11B is similar to FIG. 11A, but illustrates a front view of a medium size face cushion;

FIG. 11C is similar to FIG. 11A, but illustrates a front view of a small size face cushion;

FIG. 12A is similar to FIG. 11A, but illustrates a side view of the large size face cushion;

FIG. 12B is similar to FIG. 12A, but illustrates a side view of a medium size face cushion; and

FIG. 12C is similar to FIG. 12A, but illustrates a side view of a small size face cushion.

### DESCRIPTION OF EXAMPLE EMBODIMENTS

Example embodiments of a respiratory mask assembly for use in a continuous positive airway pressure system that incorporate aspects of the present invention are shown in the drawings. It is to be appreciated that the shown examples are not intended to be a limitation on the present invention. For example, one or more aspects of the present invention can be utilized in other embodiments and even other types of devices. It is to be appreciated that the various drawings are not necessarily drawn to scale from one figure to another nor inside a given figure, and in particular that the size of the components are arbitrarily drawn, except where otherwise noted, for facilitating the reading of the drawings. In the following description, for purposes of explanation, numerous specific details are set forth in order to provide a thorough understanding of the present invention. It may be evident, however, that the present invention may be practiced without these specific details.

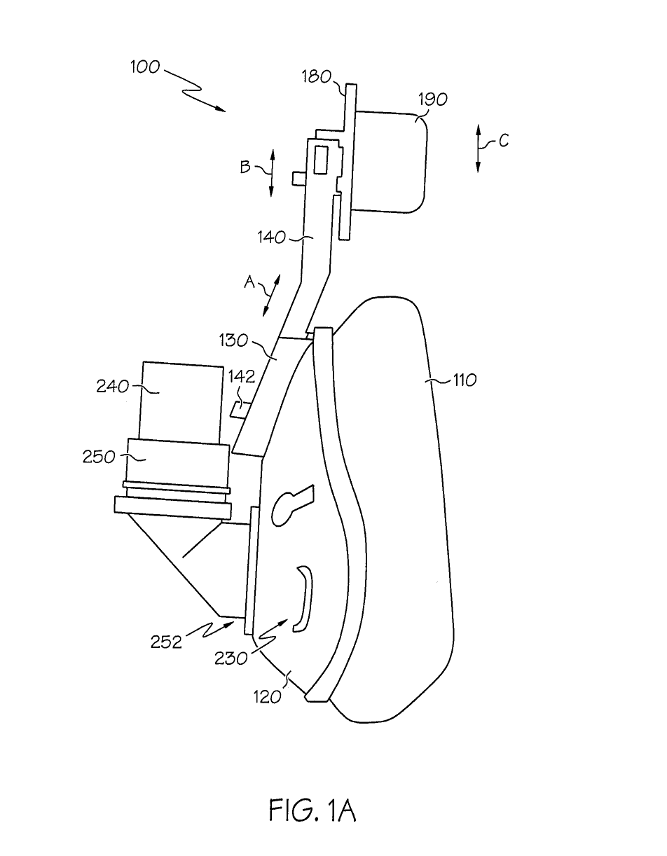

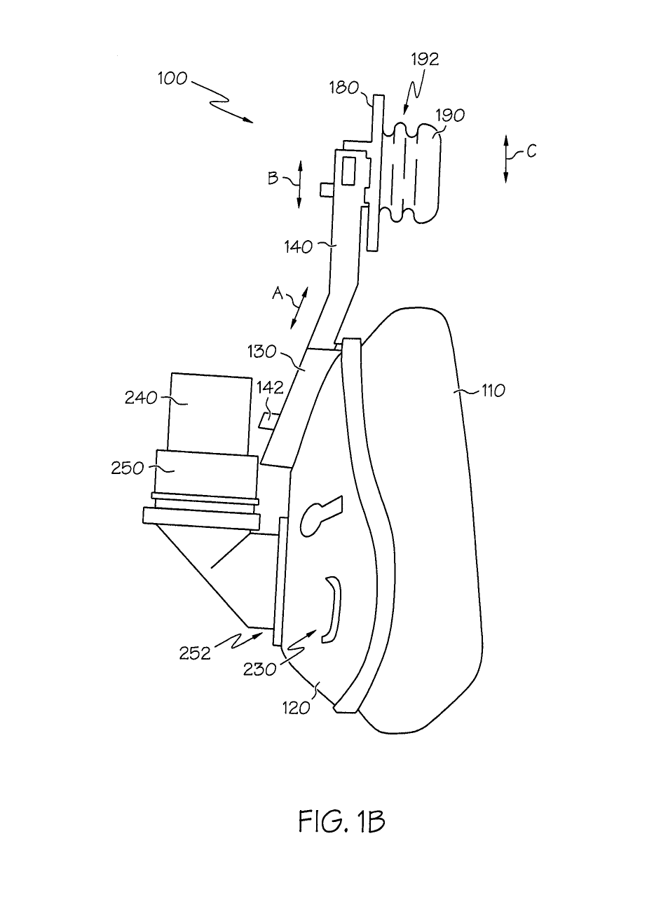

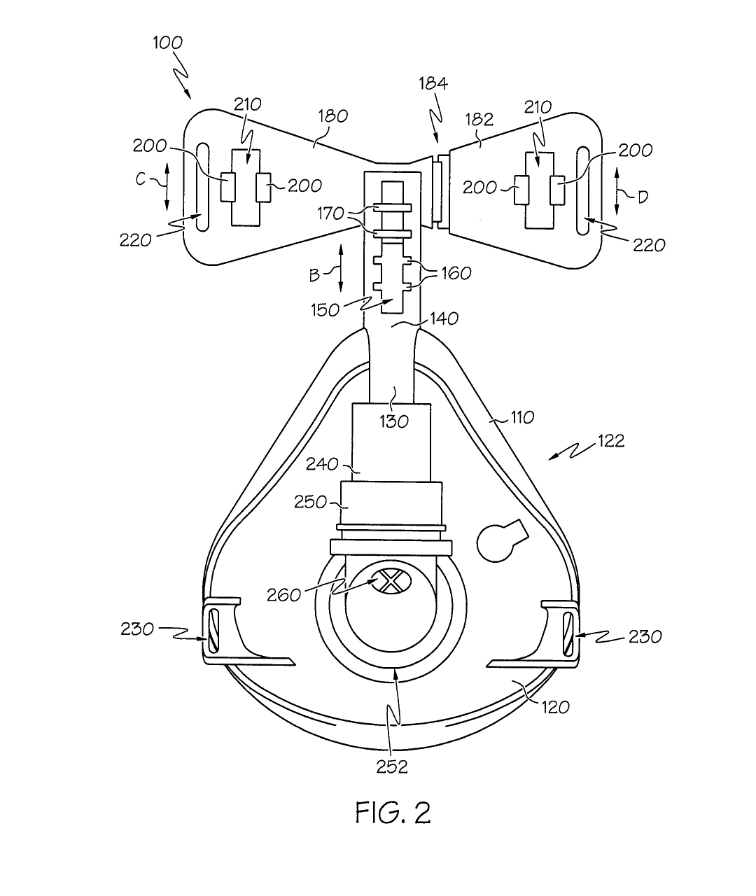

Turning to the shown example of FIGS. 1A-2, an example respiratory mask assembly 100 is illustrated. The respiratory mask assembly 100 includes a flexible cushion 110 that forms an enclosure around a mouth and/or nose of a patient and a frame 120 that is removably coupled to the flexible cushion 110. The cushion 110 can be manufactured from an elastomeric material such as polyurethane, silicone, or any other suitable material, and can have a generally triangularly-shaped outer-periphery 122 so as to fit the contours of a patient's face, though it can also have various other geometries. The generally triangularly-shaped outer-periphery 122 can trace a triangular profile similar to that of a geometric triangle (e.g., scalene, isosceles, or equilateral triangle), though it can also include other geometry. For example, any or all of the sides of the triangularly-shaped outer-periphery 122 can include inwardly-extending (e.g., depressions) or outwardly-extending (projections) portions. Further, the corners of the various sides can be sharp (e.g., pointed) or curved (e.g., rounded).

The cushion 110 can be a nasal mask configured to fit the patient's face from above the nose to a mustache area, located between the nose and the lips, or a full face mask configured to fit the patient's face from above the nose to below the mouth (e.g., a chin region). The frame 120 can be manufactured from a generally rigid material, such as a polycarbonate frame, or any other type of suitable material (e.g., various plastics, hard rubbers, metals, etc.), and can also have a generally triangularly-shaped outer-periphery similar to that of the cushion 110 discussed above. The frame 120 can be configured such that it can be removably attached to a plurality of different sized cushions, such as nasal mask cushions of various sizes as well as full face mask cushions of various sizes, as will be discussed more completely herein. In addition or alternatively, the mask can include a plurality of differently sized shells, such as one for accommodating a plurality of sizes of nasal masks and another for accommodating a plurality of sizes of full face masks. Accordingly, a customer can order the CPAP mask shipped with various sized cushions thereby allowing the customer to fit himself/herself. Bayonet type straps or the like can be used to couple the cushion to the shell, though various other couplers can also be used, as will be discussed more completely herein. However it is to be appreciated that any suitable structure for removably securing one of a plurality of different sized cushions to the shell can be utilized and is contemplated as falling within the scope of the present invention.

The frame 120 can also include a support member 130 extending from the frame 120 for receiving a forehead support arm 140. The support member 130 can be angled relative to the frame 120 and includes a slotted portion (not shown) having a plurality of notches or the like therein. The notches in the support member 130 correspond with one or more projections 142 extending from a bottom portion of the forehead support arm 140. The bottom portion of the forehead support arm 140 is also generally angled to correspond with the support member 130. Thus, the forehead support arm 140 can be adjusted in a substantially diagonal manner with respect to the frame 120 along the direction of arrow A. It is to be appreciated that adjustment in a diagonal manner permits simultaneous vertical and horizontal adjustment, the degree of movement in either direction being dependent upon the angle of the support member 130. In addition or alternatively, the forehead support arm 140 can include the slotted portion and plurality of notches therein and the support member 130 could include the one or more projections to adjust the position of the forehead support arm with respect to the frame 120. It is to be appreciated that various other suitable configurations for adjusting the position of the forehead support arm with respect to the shell can also be employed.

Likewise, as more clearly shown in FIG. 2, the forehead support arm 140 includes a substantially vertical top portion. The top portion can include a slot 150 having a plurality of notches 160 formed in the sides of the slot 150. The notches 160 can be configured to correspond with outwardly extending projections 170 of a forehead support flange 180. Thus, the forehead support flange 180 can be vertically adjustable along the slot 150 with respect to the forehead support arm 140 (e.g., in the direction of arrow B). Again, it is to be appreciated that various other suitable structures for vertically adjusting the forehead support flange 180 with respect to the forehead support arm 140 can be used.

The forehead support flange 180 can be a generally flange like structure adapted to support one or more forehead cushions 190. As shown in FIG. 1A, the forehead cushion(s) 190 can be manufactured from an elastomeric material such as polyurethane, silicone, or various other suitable materials and is adapted to contact the forehead of the patient when the mask 100 is in place. As shown in FIG. 1B, an alternative forehead cushion 190 can optionally include a compression spring structure 192 to provide greater comfort to the patient. Thus, the compression spring structure 192 can permit the cushion 190 to be adjustable in length between a contracted state and an expanded state to provide greater patient comfort. Further included on the forehead cushion 190 are one or more flexible projections 200. The projection(s) 200 can be attached to the forehead cushion 190 to form a unitary body.

The projections(s) 200 can be attached in various manners, such as by fasteners, adhesives, couplers, or the like, or can even be formed with the forehead cushion 190. The projection(s) 200, when compressed, are configured to fit through and slide within at least one slot 210 provided through the forehead support flange 180. In the shown example, the support flange 180 includes two slots 210 therein, each of the slots 210 supporting two cushion projections 200. Accordingly, the projections 200 can be slid up and down within the slot(s) 210 making the forehead cushion 190 infinitely vertically adjustable with respect to the forehead support flange 180 (e.g., along the direction of arrow C). That is, the fore head cushion 190 is infinitely vertically adjustable relative to the forehead support flange 180 within a range, the geometry of the slot 210 defining the range of vertical adjustability. When the projections 200 are not compressed, the forehead cushion 190 stays in position via a friction force or the like between the projections 200 and the support flange 180. Accordingly, the forehead support flange 180 can be vertically and horizontally adjusted along the direction of arrow A, and further vertically adjusted along the direction of arrow B. In addition or alternatively, each of the forehead cushions 190 can be individually vertically adjusted along the direction of arrow C to provide a more customized fit to a patient's face. It is to be appreciated that the forehead support flange 180 and the forehead cushion(s) 190 can also include similar structure adapted to permit the forehead cushion(s) 190 to be horizontally adjustable relative to the support flange 180.

In addition or alternatively, the mask assembly 100 can include a plurality of moveable forehead support flanges 182, each being coupled to at least one forehead cushion 190. Each of the plurality of moveable forehead support flanges 182 can be adapted to be vertically adjustable relative to the forehead support arm 140 (e.g., along the direction of arrow D). In one example, the moveable forehead support flange 182 can be coupled to the forehead support arm 140 by way of a sliding vertical connection 184, such as a tongue and groove type connection, pin and cylinder type connection, rails, or the like. For the sake of clarity, only one movable forehead support flange 182 is shown, though multiple forehead support flanges 182 (e.g., one for each side or even multiples for each side) can be used. The relative position of the moveable support flange 182 relative to the support arm 140 via the sliding vertical connection 184 can be maintained in various manners, such as by fasteners, a selective locking connection (e.g., projection in slots or the like, such as the slots 160 and projections 170 previously described), a friction fit, or the like. Further, at least one of the forehead cushions 190 coupled to the moveable support flange 182 can be adapted to be vertically adjustable (as previously described herein) relative to the movable flange 182 that it is coupled to. Accordingly, the movable forehead support flange 182 can be individually vertically adjusted along the direction of arrow D, and the forehead cushion 190 can be individually vertically adjusted along the direction of arrow C, to provide a even greater customized fit to a patient's face.

Two sets of headgear strap flanges 220, 230 are provided on the adjustable mask assembly 100 to facilitate utilization of headgear straps (not shown). One set of headgear strap flanges 220 are provided in the forehead support flange 180 and the other set of headgear strap flanges 230 are provided in the frame 120. The flanges 220, 230 include apertures for receiving the headgear straps. When the mask is positioned on the face of the patient, the headgear straps fasten around the patient's head and apply pressure to the frame 120 and thus the cushion 110 as well as to the support flange 180 and forehead cushion 190, securing the mask assembly 100 against the patient's face.

A supply tube 240 is coupled to the frame 120 to deliver air pressure from a ventilation device (not shown) to a patient via flexible tubing (not shown). A swivel portion 250 can be coupled to the supply tube(s) 240 to facilitate easy manipulation of the tubing for patient comfort. In addition or alternatively, the supply tube 240 and swivel portion 250 can rotate relative to the frame 120 about an additional swivel connection 252 for additional patient comfort. The ventilation device forces a gas, such as air, through the supply tube(s) and can be provided by a continuous positive airway pressure (CPAP) machine, a bi-level positive airway pressure machine, an intermittent (non-continuous) positive pressure (IPPB) machine, or any other suitable machine to deliver air through the mask assembly 100 to the patient. For sleep apnea therapy, the mechanical ventilator will usually supply room air at a pressure of between five and fifteen centimeters of water. The room air may be supplemented with oxygen if desired by splicing an oxygen supply line into the supply hose or using a triple port connector. An air vent 260 is provided in the mask assembly 100 to minimize noise and facilitates carbon dioxide washout.

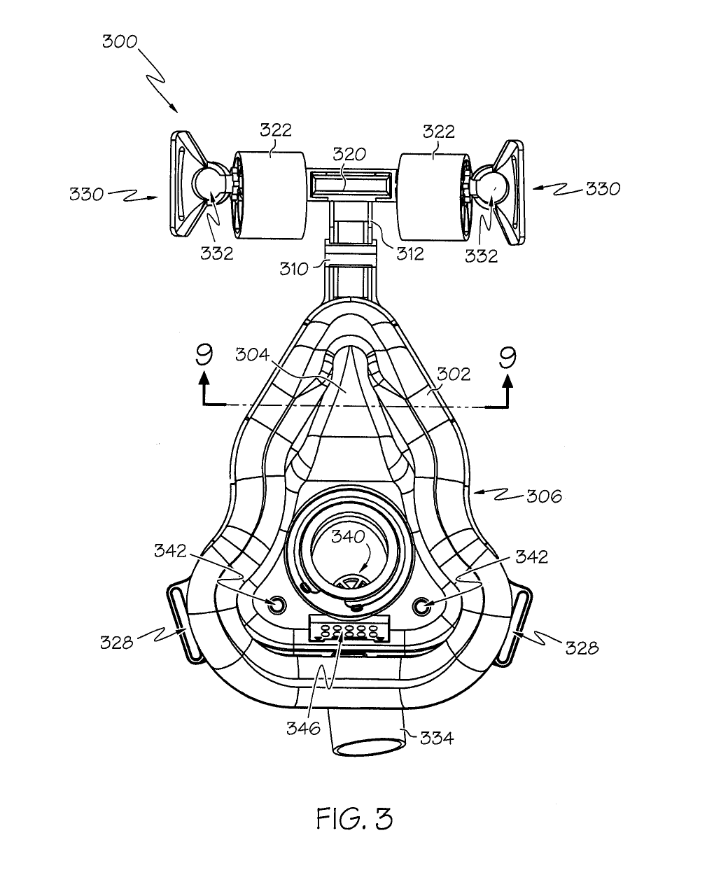

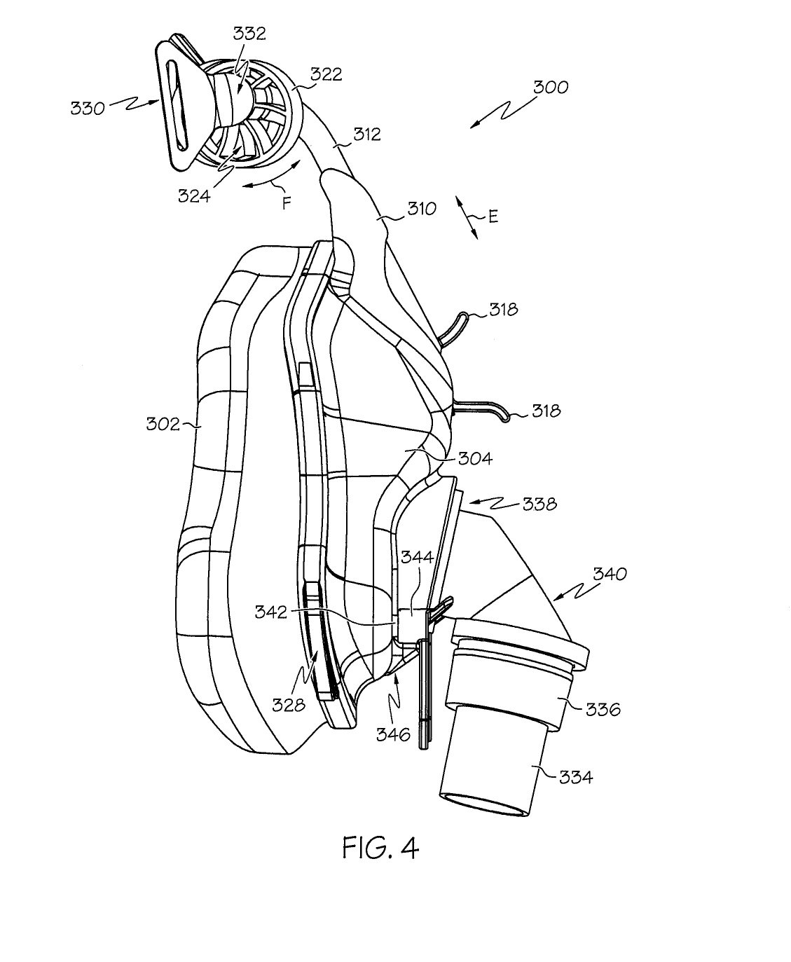

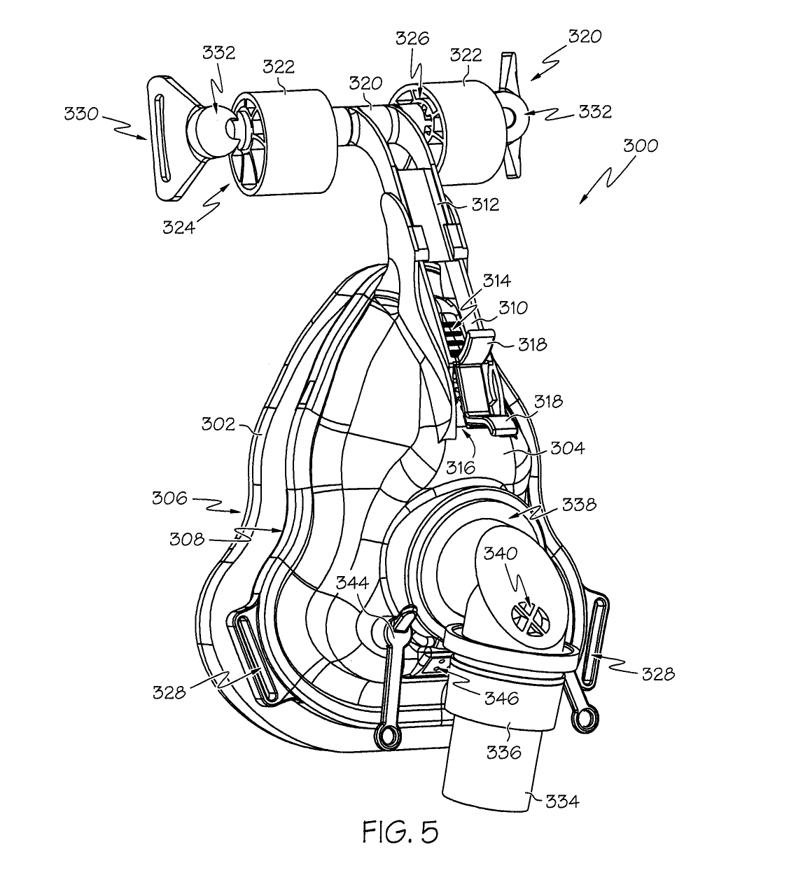

Turning now to the shown example of FIGS. 3-5, another example respiratory mask assembly 300 is illustrated. The respiratory mask assembly 300 includes a flexible cushion 302 that forms an enclosure around a mouth and/or nose of a patient and a frame 304 that is removably coupled to the flexible cushion 302. The cushion 302 is manufactured from an elastomeric material such as polyurethane, silicone, or any other suitable material, and can have a generally triangularly-shaped outer-periphery 306 so as to fit the contours of a patient's face, though it can also have various other geometries. The generally triangularly-shaped outer-periphery 306 can trace a triangular profile similar to that of a geometric triangle (e.g., scalene, isosceles, or equilateral triangle), though it can also include other geometry. For example, any or all of the sides of the triangularly-shaped outer-periphery 306 can include inwardly-extending (e.g., depressions) or outwardly-extending (projections) portions. Further, the corners of the various sides can be sharp (e.g., pointed) or curved (e.g., rounded).

The cushion 302 can be a nasal mask configured to fit the patient's face from above the nasal bridge region to a mustache area, located between the nose and the lips, or a full face mask (as shown) configured to fit the patient's face from above the nasal bridge region to below the mouth (e.g., a chin region). The frame 304 can be manufactured from a generally rigid material, such as a polycarbonate frame, or any other type of suitable material (e.g., various plastics, hard rubbers, metals, etc.). The frame 304 is configured such that it can be removably attached to a plurality of different sized cushions, such as nasal mask cushions of various sizes as well as full face mask cushions of various sizes, as will be discussed more completely herein. The frame can also have a generally triangularly-shaped outer periphery 308 similar to the outer-periphery 306 of the cushion 302.

The mask assembly 300 can further include various additional structure similar to that previously described herein. For example, the frame 304 can include a support member 310 extending from the frame 304 for receiving a forehead support arm 312. The support member 310 can be angled relative to the frame 304 and include a slotted portion having a plurality of notches 314 or the like therein that correspond with one or more projections 316 extending from a bottom portion of the forehead support arm 312. The bottom portion of the forehead support arm 312 can also include one or more handles 318 to facilitate engagement and/or release of the projections 316 with/from the notches 314. The bottom portion of the forehead support arm 312 is also generally angled to correspond with the support member 310 such that the forehead support arm 312 can be adjusted in a substantially diagonal manner with respect to the frame 304 along the direction of arrow E. It is to be appreciated that adjustment in a diagonal manner permits simultaneous vertical and horizontal adjustment, the degree of movement in either direction being dependent upon the angle of the support member 310. In addition or alternatively, the forehead support arm 312 can include the slotted portion and plurality of notches therein and the support member 310 could include the one or more projections to adjust the position of the forehead support arm with respect to the frame 304. It is to be appreciated that various other suitable configurations for adjusting the position of the forehead support arm with respect to the shell can also be employed. For example, though not shown, the forehead support arm 312 could include additional vertical or horizontal adjustment elements similar to the slot 150, notches 160, and/or projections 170 as previously described herein.

The forehead support arm 312 can further include a forehead support flange 320 adapted to support one or more forehead cushions 322. The forehead cushion(s) 322 can be manufactured from an elastomeric material such as polyurethane, silicone, or various other suitable materials, and can be adapted to contact the forehead of the patient when the mask 300 is in place. The forehead cushion(s) 322 can also optionally include a compression spring structure 324 or the like to provide greater comfort to the patient. As shown, the support flange 320 can have a generally curved geometry, such as a circular geometry. Thus, the forehead cushions 322 can have a similar generally curved geometry, such as a circular geometry, though various other geometries are also contemplated, such as elliptical, triangular, square, rectangle, polygonal, etc. As shown, the forehead cushions 322 can be generally circular and can be rotatably mounted to the support flange 320 about an axis offset a distance from a central axis.

Accordingly, rotation of a forehead cushion 322 relative to the support flange 320 (e.g., in the direction of arrow F) can cause the exterior of the forehead cushion 322 (e.g., the outer periphery thereof) to move relatively closer or relatively farther away from a patient's forehead to provide horizontal adjustability for greater comfort to the patient. In one example, the exterior of the forehead cushion 322 can be considered to circumscribe an orbit about the offset axis. Thus, the forehead cushion 322 can be infinitely horizontally adjustable, relative to a patient's forehead, through a range generally defined by the apogee (maximum distance) and perigee (minimum distance) of the exterior's orbit about the offset axis. The forehead cushions 322 can also include ratchet structure 324 or the like for cooperation with corresponding structure of the support flange 320 to provide discrete angles of rotation for the forehead cushions 322. In addition or alternatively, as described previously herein, the mask assembly 300 can include a plurality of moveable forehead support flanges (not shown) to provide individual vertical adjustability relative to the forehead support arm 312 (e.g., along the direction of arrow D) to provide an even greater customized fit to a patient's face.

Two sets of headgear strap flanges 328, 330 are provided on the adjustable mask assembly 330 to facilitate utilization of headgear straps (not shown). One set of headgear strap flanges 328 are provided in the frame 304 and the other set 330 are provided in the forehead support flange 320. As shown, the set of headgear strap flanges 330 can be adjustably coupled to the forehead support flange 320, such as by way of a ball and socket joint 332 or the like, though various other adjustable couplings can also be used. The flanges 328, 330 include apertures for receiving the headgear straps. When the mask is positioned on the face of the patient, the headgear straps fasten around the patient's head and apply pressure to the frame 304 and thus the cushion 302 as well as to the support flange 312 and forehead cushion 322, securing the mask assembly 300 against the patient's face.

A supply tube 334 is coupled to the frame 304 to deliver air pressure from a ventilation device (not shown) to a patient via flexible tubing (not shown). A swivel portion 336 can be coupled to the supply tube(s) 334 to facilitate easy manipulation of the tubing for patient comfort. In addition or alternatively, the supply tube 334 and swivel portion 336 can rotate relative to the frame 304 about a swivel connection 338 for additional patient comfort. The ventilation device forces a gas, such as air, through the supply tube(s) and can be provided by a continuous positive airway pressure (CPAP) machine, a bi-level positive airway pressure machine, an intermittent (non-continuous) positive pressure (IPPB) machine, or any other suitable machine to deliver air through the mask assembly 300 to the patient. For sleep apnea therapy, the mechanical ventilator will usually supply room air at a pressure of between five and fifteen centimeters of water.

The mask assembly 300 can also be provided with one or more vents for various reasons, such as safety or added convenience. For example, a safety vent 340 can be provided to act as an anti-asphyxia device. The safety vent 340 can include a one-way valve or the like. When the air pump is operational, the one-way valve can close off the safety vent 340 so as to enable the mask assembly 300 to remain pressurized. However, if the pump is turned off (e.g., intentionally or unintentionally) or the pressurized flow of air to the mask 300 is otherwise interrupted, the one-way valve can automatically open the safety vent 340 to permit the patient to breathe normally. In another example, the room air may be supplemented with oxygen if desired by splicing an oxygen supply line into the supply hose or using a triple port connector. As such, the mask 300 can include one or more bleed ports 342 for connecting the oxygen supply line to the mask 300. The bleed ports 342 can extend through the mask 300 to provide for a direct connection to the mask interior. In addition or alternatively, various monitoring devices such as sensors (e.g., pressure, flow, temperature, gas analysis, etc.) or the like can be connected to the bleed ports 342. Further, a cap 344 or the like can be provided to each of the bleed ports 342 for sealing the bleed ports 342 when not in use. The cap 344 can also include a strap or the like for attachment to the mask 300. Any or all of the bleed ports 342 can include one-way valves or the like. In yet another example, one or more additional air vent(s) 346 can be provided in the mask assembly 300 to facilitate carbon dioxide washout. The additional air vent 346 can provide generally continual venting of carbon dioxide and other exhaled gases. As shown, the additional air vent 346 can be provided with a plurality of open holes to permit continual venting to the atmosphere outside of the mask 300, though it can also include a one-way valve or the like.

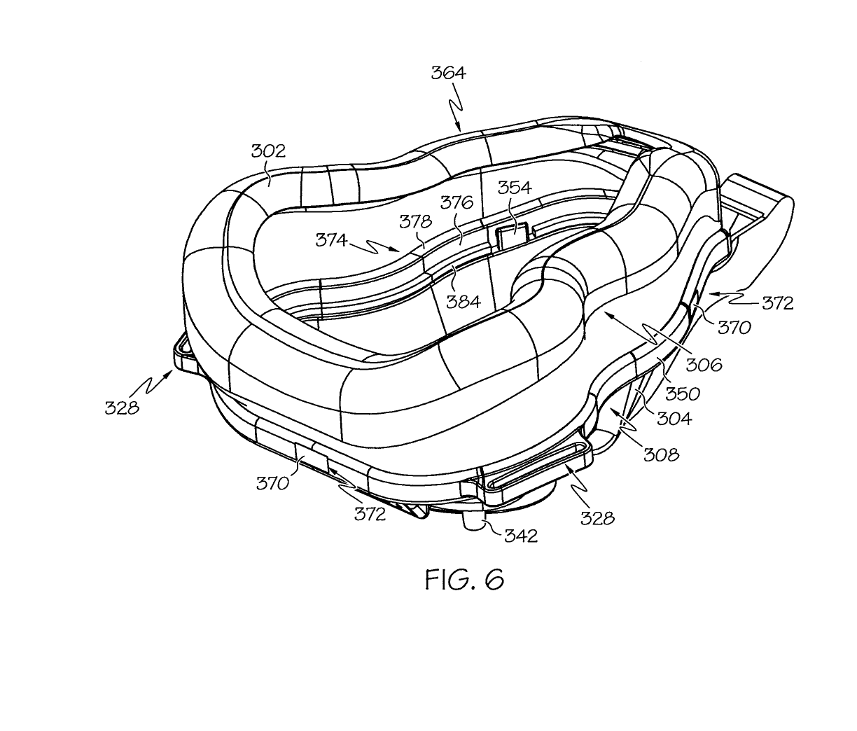

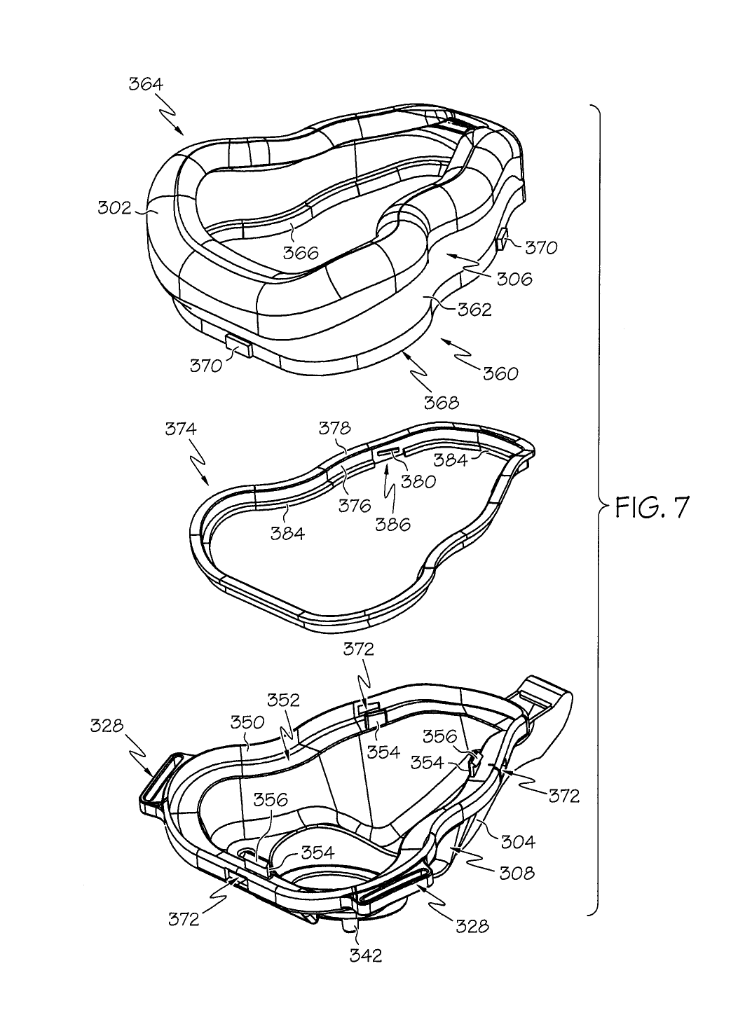

Turing now to the examples shown in FIGS. 6-10, an example connection between the face cushion 302 and the frame 304 will now be described. The outer periphery 308 of the mask frame 304 can include a wall 350 extending a distance therefrom, and a shelf 352 extending inwardly from the wall 350. As shown in FIG. 7, both of the wall and the shelf 350, 352 can extend about the entire outer periphery 308 of the frame 304 and can generally mimic the triangularly-shaped geometry, though it is to be appreciated that either or both of the wall and the shelf 350, 352 can have various shapes and sizes. Further, as shown, the shelf 352 is oriented generally orthogonal to the wall 350, though various other angles are also contemplated.

The shelf 352 can further include a retainer member 354 extending therefrom, such as a projection having a catch 356 disposed at a distal end thereof. As shown, the frame 304 can include a plurality of retainer members 354. For example, three retainer members 354 can be spaced about the shelf 352, two of which are located about an upper portion thereof and one of which located about a lower portion, though various numbers of retainer members 354 can be spaced variously about the shelf 352. The catch 356 can include various structures adapted to check motion, such as various hooks, latches, etc. Further, the retainer member 354 can be resiliently biased towards a generally upstanding position, such as where a proximal end of the retainer member 354 is coupled to the shelf 352 while the distal end remains free. It is to be appreciated that while the retainer members 354 are all shown fixed to the shelf 352, any or all of the retainer members 354 can also be removably attached to the shelf 352 in various manners.

The face cushion 302 can include various structures for engagement with the mask frame 304. For example, the face cushion 302 can include a frame-engaging portion 360 connected by a sidewall 362 to a face-engaging portion 364. As shown, the frame-engaging portion 360, sidewall 362, and face-engaging portion 364 can be formed together as a unitary body, though they can also be separately attached. The face-engaging portion 364 can be adapted to sealingly connect the respiratory mask assembly 300 to a wearer's face (not shown). The frame engaging portion 360 can be adapted to be supported by the shelf 352 when the face cushion 302 is coupled to the mask frame 304. As shown, a base portion 368 of the frame-engaging portion 360 can have a generally triangularly-shaped geometry or shape generally the same as that of the shelf 352 such that the base portion 368 and the shelf 352 extend in face-to-face adjacency to sealingly connect the face cushion 302 to the mask frame 304.

The frame-engaging portion 360 can further include a ledge 366 extending a distance from the sidewall 362, such as a distance directed inwardly. The ledge 366 can extend completely or partially about an inner periphery of the face cushion 302. In the shown example, the ledge 366 extends completely around the inner periphery of the face cushion 302 and includes a triangularly-shaped geometry similar to that of the outer periphery 306. The ledge 366 can extend inwardly generally orthogonal to the sidewall 362, though it can also extend at an angle relative thereto.

The frame-engaging portion 360 can be maintained in abutment with the shelf 352 by a portion of the ledge 366 being retained by the retainer member 354. It is to be appreciated that the ledge 366 can be directly or indirectly retained by the retainer member 354. In one example, the ledge 366 can be directly retained by the retainer member 354 such that the catch 356 extends over and grips a portion of the ledge 366. For example, the ledge 366 can have a thickness slightly greater than the distance measured from the shelf 352 to the catch 356. Thus, the ledge 366 can have a compression or interference fit with the catch 356 to thereby maintain the frame-engaging portion 360 in abutment with the shelf 352. Thereafter, the retainer member 354 can be pivoted or bent backwards to release the catch 356 from the ledge 366 to subsequently release the frame-engaging portion 360 from the shelf 352.

In addition or alternatively, the cushion 302 can further include a lug 370 for engagement with the mask frame 304. The lug 370 can facilitate orientation of the cushion 302 relative to the mask frame 304, and/or can even facilitate retention of the frame-engaging portion 360 in abutment with the shelf 352. The lug 370 can extend a distance outwardly from the sidewall 362, and can be attached to or formed therewith. In addition or alternatively, the lug 370 can also extend a distance inwardly or at various angles to the frame-engaging portion 360 and/or the ledge 366 for engagement with various portions of the mask frame 304, or even additional elements. It is to be appreciated the lug 370 can also include various other geometries and can be coupled to various portions of the cushion 302.

As shown in FIGS. 6-7, the lug 370 can extend a distance outwardly from the sidewall 362 for engagement with an aperture 372 of the mask frame 304. The aperture 372 can extend partially into and/or through a portion of the wall 350 and/or the shelf 352. The aperture 372 can have a generally similar geometry to that of the lug 370, though it can also include structure to facilitate engagement with the lug 370. In one example, the aperture 372 can have a slightly smaller size than that of the lug 370 so as to create an interference fit with the lug 370. In another example, either or both of the lug 370 and the aperture 372 can have ribs, catches, or the like for engagement with corresponding features of the lug 370/aperture 372. Both of the cushion and the mask frame 302, 304 can include the same number of lugs or apertures 370, 372, respectively, such as three each (as shown), though either or both of the cushion and the mask frame 302, 304 can include various numbers of each in various combinations. Further, the mask frame 304 can include a lug, while the cushion 302 can include an aperture. Thus, engagement of the lugs 370 with the apertures 372 can facilitate orientation of the cushion 302 relative to the mask frame 304, or even retention of the frame-engaging portion 360 in abutment with the shelf 352.

In addition or alternatively, the mask assembly 300 can also include a collar 374 to further facilitate the attachment of the cushion 302 to the mask frame 304. The collar 374 can be formed of a generally rigid material, such as plastic, hard rubber, metal, or the like, and can have a generally triangular geometry similar to that of the cushion 302 and/or mask frame 304. As shown in the exploded view of FIG. 7, the collar 374 can be disposed between the cushion 302 and the mask frame 304, and generally inwardly of the cushion 302. That is, as shown in FIG. 8, the collar 374 can have a size and geometry that is generally similar to an inner peripheral edge of the cushion 302 such that the collar 374 can abut the ledge 366 of the frame-engaging portion 360.

The collar 374 can include a side member 376 and a top member 378 extending a distance therefrom. The top member 378 can extend outwardly from the side member 376 and generally orthogonal thereto, though it can also extend inwardly and/or at various angles. As shown, the size and geometry of the top member 378 can be generally similar to that of the shelf 352. The side member 376 can also include various structures for engagement with either or both of the cushion 302 and the mask frame 304. In one example, the side member 376 can include an anchor 380 or the like. As shown, the anchor 380 can include a projection or the like extending a distance inwardly of the side member 376. The anchor 380 can be adapted for removable or non-removable engagement with the retainer member 354, such as with the catch 356. Thus, when assembled, the catch 356 of the retainer member can extend over and capture the anchor 380. Accordingly, as shown, the collar 374 can include the same number and orientation of anchors 380 as retainer members 354 of the mask frame 304, though it can also include various numbers of anchors 380 arranged in various other manners. In one example, a single continuous anchor 380 can be used. It is to be appreciated that the anchor 380 can include various other structures for engagement with corresponding structure of the retainer members 354. In various examples, the anchor 380 can include a catch, latch, pinned connection, aperture, fastener, or the like, though various other connectors are also contemplated. Further, any or all of the lugs 370 can be adapted to extend from the sidewall 362 for engagement with an aperture or the like (not shown) provided on the collar 374.

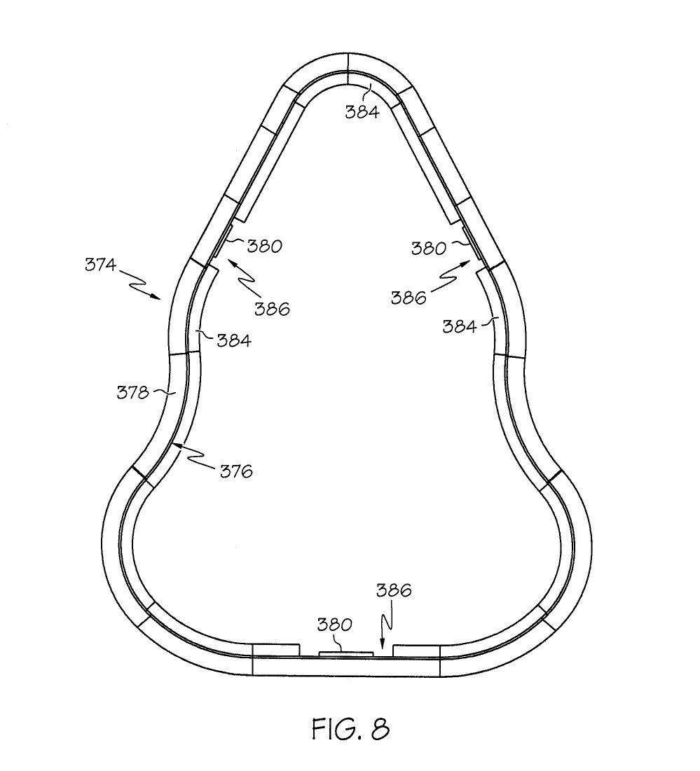

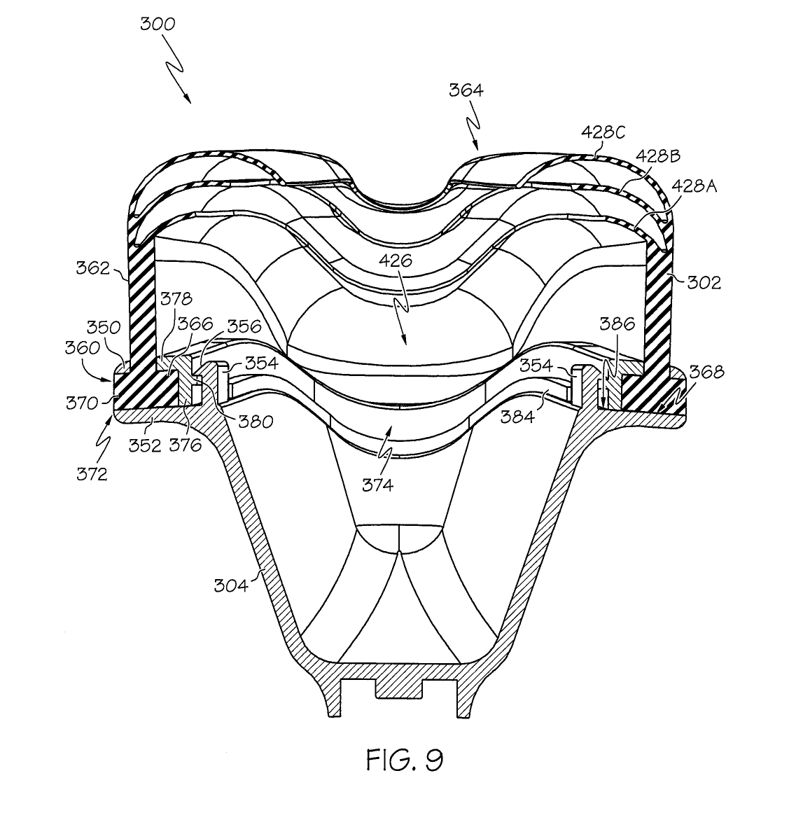

Turning briefly to the example shown in FIG. 9, a sectional view of an example coupling of the cushion 302 to the mask frame 304 is illustrated. As shown, the base portion 368 of the frame-engaging portion 360 is in abutment with the shelf 352 of the mask frame 304. The top member 378 of the collar 374 is engaged with the ledge 366, and the anchor 380 is engaged with the retainer member 354 to sealingly connect the cushion 302 to the mask frame 304. As previously described, the ledge 366 can have a slightly larger size (e.g., slightly thicker) than the distance between the top member 378 and the shelf 352 such that the engagement of the collar 374 to the mask frame 304 applies a compression or pressure to the frame-engaging portion 360. As such, in one example, a relatively tight seal can be maintained between the cushion 302 and the mask frame 304. In another example, the aforedescribed connection can maintain an airtight seal between the cushion 302 and the mask frame 304. As can be appreciated, the ledge 366 can alternatively have approximately the same size as the distance between the top member 378 and the shelf 352 such that the engagement of the collar 374 to the mask frame 304 applies little or no compression or pressure to the frame-engaging portion 360.

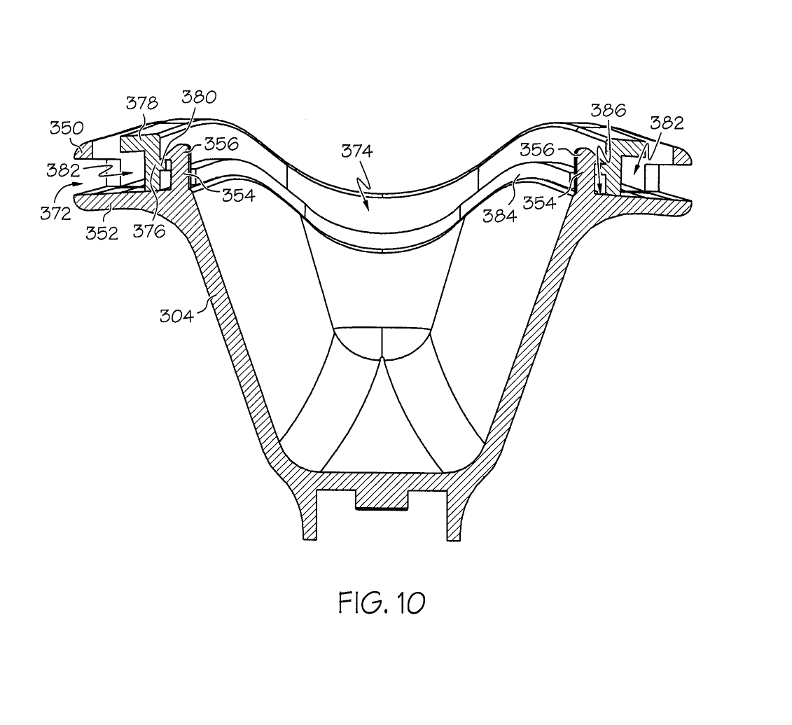

Independent of the pressure or compression applied to the frame-engaging portion 360, engagement of the collar 374 with the mask frame 304 is adapted to retain the cushion 302 so as to inhibit, or even prevent, inadvertent removal of the cushion 302 from the mask frame 304. Turning briefly now to FIG. 10, the sectional view of FIG. 9 is shown with the cushion 302 removed for greater clarity. As can be seen, a passage 382 is formed upon engagement of the collar 374 with the mask frame 304 for retaining the frame-engaging portion 360 of the cushion 302. The passage 382 can be defined by at least the wall 350 and shelf 352 of the mask frame 304, and the side member 376 and top member 378 of the collar 374, though various other elements can also provide definition. In the shown example, the passage 382 is generally continuous and has a geometry that generally follows the triangular shape of the shelf 352 about the mask frame 304. As can be appreciated, the geometry of the passage 382 can vary with the geometries of the mask frame 304 and the collar 374. Thus, the passage 382 can be completely enclosed, or as shown, can have various openings, such as those resulting from the aperture 372 of the wall 350 and/or a spacing gap located between the top member 378 of the collar 374 and the wall 350. The passage 382 can also be continuous or discontinuous, and as such a single or even multiple passages can be formed. Accordingly, the passage 382 is formed upon (e.g., simultaneously with) or after (e.g., subsequent to) attachment of the collar 374 to the mask frame 304 to capture and/or encapsulate the frame-engaging portion 360 of the cushion 302 to maintain abutment thereof with the mask frame 304.

The collar 374 can also include various other structures. For example, the collar 374 can also include a base member 384 extending a distance from the side member 376. As shown, the base member 384 can extend a distance inwardly from the side member 376 to provide additional support for the collar 374 upon the shelf 352 of the mask frame 304. The base member 384 can have a generally triangularly-shaped geometry similar to that of the side member 376 of the collar 374, though it can also have various other geometries. In one example, the base member 384 can include one or more apertures 386 so as to create space for the retainer members 354 of the mask frame 304. That is, as shown in FIGS. 9-10, the apertures 386 can provide the space required to permit the retainer members 354 to engage the anchors 380 of the collar 374. As such, the collar 374 can include the same number of apertures 386 as retainer members 354 and/or anchors 380, though various numbers of apertures 386 having various geometries can also be used.

As stated previously, the frame 304 can be configured such that is can be removably attached to a plurality of different sized cushions 302, such as nasal mask cushions of various sizes as well as full face mask cushions of various sizes. As can be appreciated, different patients will often have differently sized and/or shaped faces. Thus, in order to maintain a seal, and most especially an airtight seal, between the cushion 302 and a patient's face (not shown), a plurality of cushions can together accommodate a large percentage of the patient population. Thus, the plurality of cushions can each be provided with similar frame-engaging portions 360 and different face-engaging portions 364. For example, each of the differently face-engaging portions 364 can have at least one parameter that is different from the others to provide greater patient comfort and greater sealing.

As also stated previously, CPAP masks are adapted be used with a ventilation device (not shown) that forces a gas, such as air, through supply tube(s) by way of a continuous positive airway pressure (CPAP) machine, a bi-level positive airway pressure machine, an intermittent (non-continuous) positive pressure (IPPB) machine, or any other suitable machine to deliver air through the mask assembly 300 to the patient. For sleep apnea therapy, the mechanical ventilator will usually supply room air at a pressure of between five and fifteen centimeters of water. Thus, the face-engaging portion 364 is adapted to sealingly connect the respiratory mask to a wearer's face so as to inhibit leakage of pressurized fluid supplied by a continuous positive airway pressure pump. In one example, the different face-engaging portions 364 can each be adapted to form a generally airtight seal to sealingly connect the respiratory mask to differently sized and/or shaped patient's faces. As such, a single mask frame can be provided to a patient along with multiple face cushions of different sizes to permit the patient to determine the most comfortable fit.

Turning now to FIGS. 11A-11C, three example cushions 400, 402, 404 are illustrated. As can be seen, the example of FIG. 11A illustrates a cushion 400 (hereinafter the “large cushion”) having a relatively larger face-engaging portion 406, FIG. 11B illustrates a cushion 402 (hereinafter the “medium cushion”) having a face-engaging portion 408 relatively smaller than that of the large cushion 400, and FIG. 11C illustrates a cushion 404 (hereinafter the “small cushion”) having a face-engaging portion 410 relatively smaller than either of the large and medium cushions 400, 402. It is to be appreciated that the cushions 400, 402, 404 are each similar to the cushion 302 previously described herein, and that all such previous disclosure is incorporated herein by reference thereto. It is also to be appreciated that more or less than three cushions 400, 402, 404 can be used with the mask frame 304, each of the cushions having varying sizes, geometries, features, etc.

For the sake of brevity, the general geometry of only the large cushion 400 will now be described, with the understanding that such description applies equally to both of the medium and small cushions 402, 404, though any or all of the cushions 400, 402, 404 can have differing features. The face-engaging portion 406 includes a nasal bridge region 420 positioned in use above the patient's nose at a bridge portion thereof, and a chin region 422 generally positioned about a patient's chin below the lips. A cheek region 424 separates the nasal bridge region 420 and the chin region 422. As shown, the outer peripheral edge of the cushions 400, 402, 404 can extend generally inwards (e.g., a concave deviation or the like) about the cheek region 424 so as to generally avoid contact with a fatty cheek area of a patient's face to provide greater comfort. Thus, the cheek region 424 more closely follows the contour of a patient's nose and away from the fatty cheek area. The face-engaging portion 406 is generally flexible to accommodate a plurality of different facial contours.

In use, the patient's face (e.g., nose and lips) is received through an aperture 426 into a chamber within the body of the cushion 400. The face-engaging portion 406 thus contacts both a surface of the patient's nose, including around the sides and over the bridge of the nose, and a portion of the patient's face in both of the chin and cheek regions. The shape of the face-engaging portion 406 is particularly suited to seal the region of the facial contour that is the crease between the sides of the nose and the face. The nasal bridge region 420 of the cushion 400 can include a strengthening portion to mitigate buckling of the nasal bridge region 420 when the headgear straps apply tension on the mask assembly 300.

Returning briefly to FIG. 9, in one example, near the chin region 424 of there are three membranes integral with the side wall 362 and extending therefrom, the membranes being denoted as 428A, 428B, and 428C. In one example, the membrane 428A is the thickest of the three membranes at the chin region 424, while the membrane 428B is thinner than the membrane 428A and thicker than the outermost membrane 428C. In another example, the thicknesses can be generally similar, though it is to be appreciated that the thickness of any of the membranes can vary relative to the others. The membranes 428A, 428B, 428C all have flexibility and can deform when a force is applied against the cushion 400 when the cushion is in place against the patient's face. The use of three or more membranes is an improvement over the use of two membranes commonly used and seems to provide significant advances in comfort for the patient. The thicknesses of the membranes 428A, 428B, 428C can vary between the chin region 424 and the nasal bridge region 420, or alternatively, the thicknesses can be generally similar. In one example, at the chin region 424 the membranes can have different thicknesses with, for instance membrane 428A being 0.06 inches thick, the membrane 428B being about 0.03 inches thick and membrane 428C being about 0.02 inches thick, though various other thicknesses are contemplated. The use of a triple membrane system in combination with the varying thickness in the membrane can provide not only a soft feeling cushion but also an improved seal. It is to be appreciated that, alternatively, any of the cushions 400, 402, 404 can have only a single membrane, or various other numbers of membranes.

Turning back to FIGS. 11A-11C, each of the different face-engaging portions 406, 408, 410 can have at least one parameter that is different from the others to provide greater patient comfort and greater sealing. In one example, each cushion can have an outer membrane (e.g., outer membrane 428C) having a face-sealing surface area adapted to sealingly connect the respiratory mask to a wearer's face, the face-sealing surface area of each of the plurality of cushions 400, 402, 404 being generally different from that of the other cushions 400, 402, 404 so as to accommodate various face geometries of various patients. The face-sealing surface area can include, for example, the portion of the outer membrane 428C that is in contact with a patient's face to provide a seal between the face-receiving aperture 426 and the outside environment to inhibit leakage of air therefrom. It is to be appreciated that the face-sealing surface area can also include any portion of either or both of the other membranes 428A, 428B that provide a seal between a patient's face and the outside environment. It is also to be appreciated that the face-sealing surface area can also include the entire surface area of any or all of the membranes 428A, 428B, 428C.

As can be seen from FIGS. 11A, 11B, and 11C, the face-sealing surface area of each of the plurality of cushions 400, 402, 404 generally decreases from FIG. 11A through FIG. 11C. That is, the large cushion 400 has a face-sealing surface area generally greater than that of the medium cushion 402, which in turn has a face-sealing surface area generally greater than that of the small cushion 404. It is to be appreciated that the cushions 400, 402, 404 are shown schematically in FIGS. 11A-11C and are not drawn to exact scale. However, FIGS. 11A-11C are intended to generally illustrate the relative size differences between the cushions 400, 402, 404.

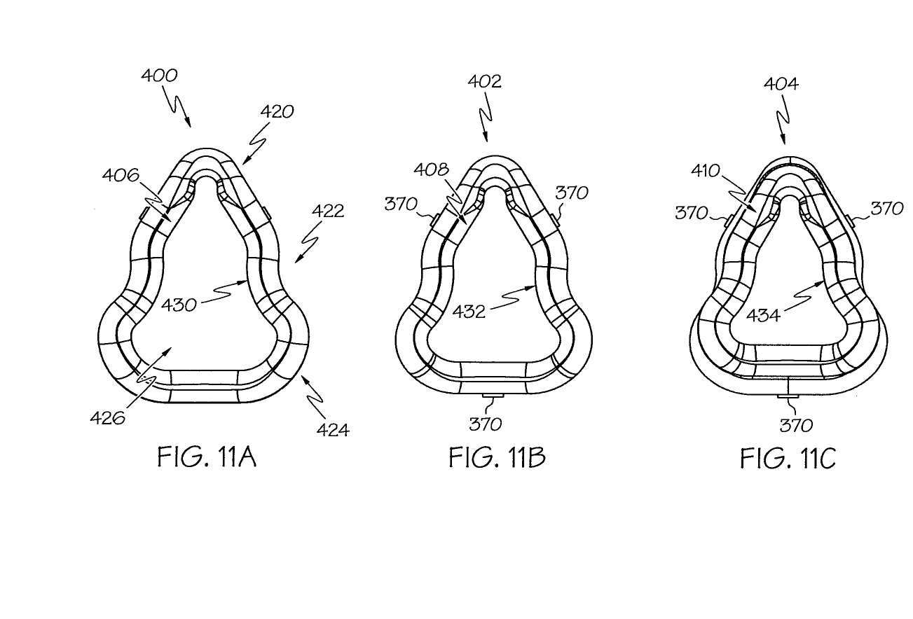

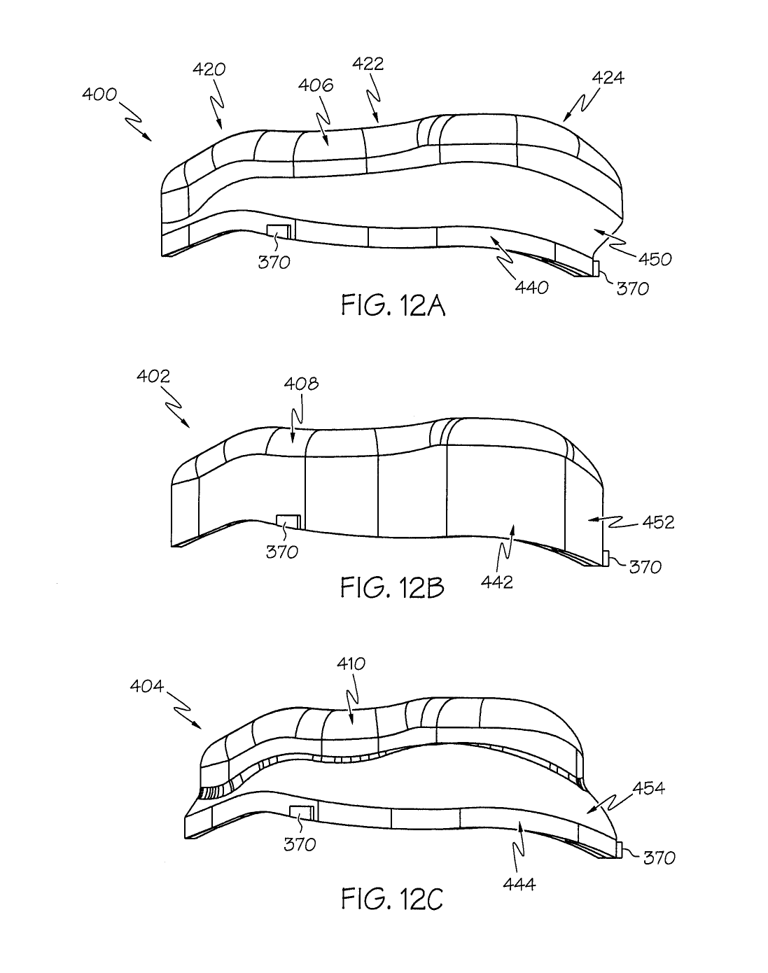

The relative reduction in face-sealing surface area can be provided in various manners. In one example, as shown in FIGS. 12A-12C, the face-engaging portions 406, 408, 410 can be increased or decreased relative to each other in various directions (e.g., along any or all of the three common major axes X, Y, Z). In another example, as shown in FIGS. 11A-11C, the outer membrane 428C can include a face-sealing perimeter 430 adapted to sealingly connect the cushion 400 to a patient's face. The face-sealing perimeter 430 of each of the plurality of cushions 400, 402, 404 can be generally different from that of the other cushions 400, 402, 404 so as to accommodate various face geometries of various users. That is, the large cushion 400 can have a face-sealing perimeter 430 generally greater than the face-sealing perimeter 432 of the medium cushion 402, which in turn is generally greater than the face-sealing perimeter 434 of the small cushion 404. It is to be appreciated that the face sealing perimeter 430, 432, 434 can include the innermost perimeter of the outer membrane(s) 428C, though it can also be located variously about the surface of the outer membrane(s) 428C depending upon the portion of the membrane(s) that seals against a patient's face. As before, it is further to be appreciated that the cushions 400, 402, 404 are shown schematically in FIGS. 11A-11C and are not drawn to scale, though are intended to generally illustrate the relative size differences between the cushions 400, 402, 404.

While the face-engaging portions 406, 408, 410 of the cushions 400, 402, 404 can be generally different to accommodate various patients, the respective frame-engaging portions 440, 442, 444 can be generally similar such that each can be sealingly engaged with the same or similar mask frames 304. Though the cushions 400, 402, 404 are shown schematically and not to scale in FIGS. 12A-12C, it can be seen that the frame-engaging portions 440, 442, 444 have a generally similar size and geometry, though relatively different sizes. That is, each of the frame-engaging portions 440, 442, 444 can be of similar shape and geometry to that of the aforedescribed shelf 352 and/or collar 374 such that the same or similar collar 374 can be used to sealingly connect any of the cushions 400, 402, 404 to the same or similar mask frame 302 in such a manner as previously described herein.

Because the frame-engaging portions 440, 442, 444 can be of similar shape and geometry while the face-engaging portions 406, 408, 410 are different, the cushions 400, 402, 404 can each include varying structure for attaching the frame-engaging portions 440, 442, 444 to the face-engaging portions 406, 408, 410. For example, as shown in FIGS. 12A-12C, the sidewalls 450, 452, 454 (respectively) can be modified so as to accommodate the relatively larger or smaller cushions. In one example, the sidewall 452 of the medium cushion 402 can have a relatively nominal size generally similar to that of the associated frame-engaging portion 442. The sidewall 450 of the large cushion 400 can have a relatively larger size (e.g., expanded) than that of the associated frame-engaging portion 440, while the sidewall 454 of the small cushion 404 can have a relatively smaller size (e.g., retracted) than that of the associated frame-engaging portion 444. The modified sidewalls 450, 452, 454 can also include various other structure, sizes and/or geometries. In one example, the modified sidewalls 450, 452, 454 can act as a bellows-like structure to provide additional support and comfort to the patient. Further, any or all of the face-sealing portions 406, 408, 410, frame-engaging portions 440, 442, 444, and sidewalls 450, 452, 404 can be formed together as a unitary body, or formed separated and removably or non-removably attached to each other. Additionally, in the shown examples, each of the cushions 400, 402, 404 is alternatively engageable (e.g., interchangeable) with the mask frame 302, though it is to be appreciated that a multi-part cushion (e.g., a major cushion formed of two or more minor cushions) can also be accommodated by a suitable mask frame 302 and/or collar 374.

It is to be appreciated that various words used herein may or may not have similar structure, and that use of different words to describe similar structure is not meant to imply that such structure must necessarily be different. For example, words used such as wall, shelf, ledge, sidewall side member, top member, or the like can describe similar or different structure. Similar, words such as aperture, notch, lug, anchor, projection, retainer member, catch, or the like can also describe similar or different structure.

The invention has been described with reference to the example embodiments described above. Modifications and alterations will occur to others upon a reading and understanding of this specification. Examples embodiments incorporating one or more aspects of the invention are intended to include all such modifications and alterations insofar as they come within the scope of the appended claims.

What is claimed is:

1. A respiratory mask assembly, including:
a generally rigid mask frame having a generally triangularly-shaped outer periphery, a wall extending from the outer periphery of the frame and including an aperture, and a shelf extending inwardly from the wall and including a retainer member extending therefrom; and
a generally triangularly-shaped cushion formed of a resilient material having a frame-engaging portion connected by a sidewall to a face-engaging portion adapted to sealingly connect the respiratory mask to a wearer's face, the frame-engaging portion including a ledge extending a distance inwardly from the sidewall and a lug extending a distance outwardly from the sidewall, the frame-engaging portion being supported by the shelf and being maintained in abutment therewith by a portion of the ledge being retained by the retainer member and the lug being engaged with the aperture of the frame.

2. The respiratory mask assembly of claim 1, wherein the frame-engaging portion includes a base portion having generally the same shape as the shelf, the base portion and the shelf extending in face-to-face adjacency to sealingly connect the cushion to the mask frame.

3. The respiratory mask assembly of claim 1, wherein the retainer member includes a catch disposed at a distal end.

4. The respiratory mask assembly of claim 1, further including a generally rigid, generally triangularly-shaped collar including a side member and a top member extending a distance outwardly therefrom, the side member including an anchor, the top member being engaged with the ledge and the anchor being engaged with the retainer member to sealingly connect the cushion to the mask frame.

5. The respiratory mask assembly of claim 4, wherein a passage is formed after engagement of the collar with the mask frame for retaining the frame-engaging portion, the passage being defined by the wall and shelf of the mask frame, and the top and side members of the collar.

6. The respiratory mask assembly of claim 1, wherein the face-engaging portion is adapted to form a generally airtight seal to sealingly connect the respiratory mask to a wearer's face.

7. A respiratory mask assembly, including:
a generally rigid mask frame having a generally triangularly-shaped outer periphery, a wall extending from the outer periphery of the frame and including an aperture, and a shelf extending inwardly from the wall and including a retainer member extending therefrom;
a generally triangularly-shaped cushion formed of a resilient material having a frame-engaging portion connected by a sidewall to a face-engaging portion adapted to sealingly connect the respiratory mask to a wearer's face, the frame-engaging portion being supported by the shelf and further including a ledge; and
a generally rigid, generally triangularly-shaped collar including a side member and a top member extending a distance outwardly therefrom, the side member including an anchor, the top member being engaged with the ledge and the anchor being engaged with the retainer member to sealingly connect the cushion to the mask frame.

8. The respiratory mask assembly of claim 7, wherein a passage is formed after engagement of the collar with the mask frame for retaining the frame-engaging portion, the passage being defined by the wall and shelf of the mask frame, and the top and side members of the collar.

9. The respiratory mask assembly of claim 7, wherein the collar further includes a base member extending a distance inwardly from the side member, the base member being adapted to be supported by the shelf.

10. The respiratory mask assembly of claim 9, wherein the base member includes an aperture adapted to receive the retainer member so as to permit the retainer member to engage the anchor.

11. The respiratory mask assembly of claim 7, wherein the frame-engaging portion further includes a lug extending a distance outwardly from the sidewall.

12. The respiratory mask assembly of claim 11, wherein the lug is engaged with the aperture of the frame.

13. The respiratory mask assembly of claim 7, wherein the ledge extends a distance inwardly from the sidewall.

14. The respiratory mask assembly of claim 7, wherein the face-engaging portion is adapted to form a generally airtight seal to sealingly connect the respiratory mask to a wearer's face.

15. A respiratory mask assembly, including:
a generally rigid mask frame having a generally triangularly-shaped outer periphery;
a generally rigid, generally triangularly-shaped collar engageable with the mask frame; and
a plurality of generally triangularly-shaped cushions being formed of a resilient material, each cushion including a face-engaging portion with an outer membrane, the collar being adapted to sealingly connect the cushions to the mask frame such that each of the cushions is alternatively engageable with the mask frame.

16. The respiratory mask assembly of claim 15, wherein each cushion includes a face-engaging portion with an outer membrane having a face-sealing surface area adapted to sealingly connect the respiratory mask to a wearer's face, the face-sealing surface area of each of the plurality of cushions being generally different from that of the other cushions so as to accommodate various face geometries of various users.

17. The respiratory mask assembly of claim 15, wherein each cushion includes a face-engaging portion with an outer membrane having a face-sealing perimeter adapted to sealingly connect the respiratory mask to a wearer's face, the face-sealing perimeter of each of the plurality of cushions being generally different from that of the other cushions so as to accommodate various face geometries of various users.

18. The respiratory mask assembly of claim 15, wherein the mask frame includes a shelf and each cushion includes a frame-engaging portion adapted to be supported by the shelf and maintained in abutment therewith by the collar, the cushions having generally similar frame-engaging portions.

19. The respiratory mask assembly of claim 18, wherein the frame-engaging portions further include a ledge, and the collar further includes a side member and a top member extending a distance outwardly therefrom, the side member including an anchor, the top member being engaged with the ledge and the anchor being engaged with a retainer member coupled to the mask frame to sealingly connect the cushion to the mask frame.

20. The respiratory mask assembly of claim 18, wherein the mask frame further includes an aperture and the frame-engaging portion further includes a lug for engagement with the aperture.

21. The respiratory mask assembly of claim 15, wherein the face-engaging portion is adapted to form a generally airtight seal to sealingly connect the respiratory mask to a wearer's face.

22. A respiratory mask assembly, including:
a generally rigid mask frame;
a cushion formed of a resilient material supported by the mask frame and adapted to sealingly connect the respiratory mask to a wearer's face; and
a forehead support including a forehead support arm coupled to the mask frame, a forehead support flange coupled to the forehead support arm, and a resilient forehead cushion coupled to the support flange, the forehead cushion being adapted to be infinitely vertically adjustable within a range relative to the forehead support arm.

23. The respiratory mask assembly of claim 22, wherein the forehead cushion further includes a projection adapted to be received within a slot of the support flange, the geometry of the slot defining the range of vertical adjustability of the forehead cushion.

24. The respiratory mask assembly of claim 23, wherein the forehead cushion and the projection form a unitary body.

25. The respiratory mask assembly of claim 22, wherein the forehead cushion is adapted to be vertically adjustable relative to the support flange.

26. The respiratory mask assembly of claim 22, further including a plurality of forehead cushions, each of the forehead cushions being adapted to be vertically adjustable relative another of the forehead cushions.

27. The respiratory mask assembly of claim 26, further including a plurality of support flanges, each of the support flanges being coupled to at least one forehead cushion, each of the support flanges being adapted to be vertically adjustable relative the forehead support arm.

28. The respiratory mask assembly of claim 27, wherein at least one of the forehead cushions coupled to each support flange is adapted to be vertically adjustable relative to the support flange that it is coupled to.

29. The respiratory mask assembly of claim 22, wherein the face-engaging portion is adapted to form a generally airtight seal to sealingly connect the respiratory mask to a wearer's face.
